(19) United States
(12) Patent Application Publication
(10) Pub. No.: US 2024/0197351 A1
(43) Pub. Date: Jun. 20, 2024

(54) SURGICAL INSTRUMENT WITH INCREASED ACTUATION FORCE

(71) Applicant: Distalmotion SA, Epalinges (CH)

(72) Inventor: Ricardo Daniel Rita BEIRA, Lausanne (CH)

(21) Appl. No.: 18/592,220

(22) Filed: Feb. 29, 2024

Related U.S. Application Data
(63) Continuation of application No. 17/664,412, filed on May 22, 2022, now Pat. No. 11,944,337.
Application No. 17/664,412 is a continuation of application No. 17/032,631, filed on Sep. 25, 2020, now Pat. No. 11,337,716.
Application No. 17/032,631 is a continuation of application No. 15/756,037, filed on Feb. 27, 2018, now Pat. No. 10,786,272.
(60) Provisional application No. 62/211,019, filed on Aug. 28, 2015.

(51) Int. Cl. A61B 17/29; A61B 17/00; A61B 17/32; A61B 17/3201; A61B 17/34; A61B 34/00; A61B 34/30

(52) U.S. Cl. CPC A61B 17/29; A61B 17/320016; A61B 17/3201; A61B 17/3478; A61B 34/71; A61B 2017/00314; A61B 2017/00323; A61B 2017/00367; A61B 2017/2913; A61B 2017/2918; A61B 2017/2919; A61B 2017/2933; A61B 2017/2934; A61B 2017/2937; A61B 2034/305

(57) ABSTRACT

A surgical instrument with improved end-effector gripping force. The instrument comprises a shaft, which may be inserted into a body of a patient. The articulated end-effector is mounted on the distal extremity of the instrument shaft and comprises a plurality of links interconnected by a plurality of joints, whose movements are remotely actuated by the surgeon's hands. This remote actuation is accomplished through mechanical transmission, mainly along flexible elements, which are able to deliver motion from a set of actuation elements, placed at a proximal extremity of the shaft, to the instrument's articulated end-effector. The articulated end-effector further comprises one or more cam-and-follower mechanisms that are able to amplify the force transmitted by the flexible elements so that the actuation force at the instrument jaws is maximized and the tension on the transmission elements minimized, thus increasing the fatigue resistance and life of the instrument.

### CROSS-REFERENCE TO RELATED APPLICATIONS

This application is a continuation of U.S. patent application Ser. No. 15/756,037, filed Feb. 27, 2018, now U.S. Pat. No. 10,786,272, which is a national phase of International PCT Patent Application Serial No. PCT/IB2016/001286, filed Aug. 29, 2016, which claims priority to U.S. Provisional Patent Application Ser. No. 62/211,019, filed Aug. 28, 2015, the entire contents of each of which are incorporated herein by reference.

### FIELD OF THE INVENTION

The present invention relates to the field of remotely actuated mechanical systems, more particularly to endoscopic or minimally invasive mechanisms, and most particularly to remotely actuated minimally invasive surgical instruments. More specifically, this invention relates to minimally invasive articulated surgical instruments such as graspers, needle holders, and scissors, wherein the orientation of end-effectors in relation to the instrument shaft is able to be controlled. Most specifically, the invention relates to mechanisms wherein the actuation and orientation of the instrument's distal end-effector is remotely performed, from the proximal to the distal extremity of the instrument shaft, by mechanical transmission elements.

### BACKGROUND OF THE INVENTION

Open surgery is still the standard technique for most surgical procedures. It has been used by the medical communication for several decades and consists of performing surgical tasks by making a relatively long incision in the abdomen or other body cavity or area, through which traditional surgical tools are inserted. However, due to the long incision, this approach is extremely invasive for the patient, resulting in substantial blood loss during the surgery and long and painful recovery periods in an in-patient setting.

In order to provide an alternative to the invasiveness of open surgery, laparoscopy, a minimally invasive technique, was developed. Instead of a single long incision, one or more smaller incisions are made in the patient through which long and thing surgical instruments and endoscopic cameras are made in the patient through which long and thin surgical instruments and endoscopic cameras are inserted. Because of the low degree of invasiveness, laparoscopic techniques reduce blood loss and pain while also shortening hospital stays. When performed by experienced surgeons, these techniques can attain clinical outcomes similar to open surgery. However, despite the above-mentioned advantages, laparoscopy requires advanced surgical skills to manipulate the rigid and long instrumentation through small incisions in the patient. As such, adoption rates for minimally invasive techniques in complex procedures are lower than would be desirable.

Traditionally, laparoscopic instruments, such as graspers, dissectors, scissors and other tools, have been mounted on straight shafts. These shafts are inserted through small incisions into the patient's body and, because of that, their range of motion inside the body is reduced. The entry incision acts as a point of rotation, decreasing the freedom for positioning, actuating, articulating and orientating the instruments inside the patient. Also, the use of straight-shafted instruments prevents bending or articulation inside the surgical space. Therefore, due to the challenges facing traditional minimally invasive instrumentation, laparoscopic procedures are mainly limited to use in simple surgeries, while only a small minority of surgeons is able to use them in complex procedures.

Accordingly, there is a clear need for providing distal articulations to effector elements of laparoscopic instruments, allowing the distal end-effector elements to be articulated with respect to the longitudinal axis of the instrument shaft. This enables the surgeon to reach the tissue of interest at a full range of angles, including oblique angles, with respect to the longitudinal axis of the shaft. In addition, the instrument should be able to fully operate its effector elements at such angulations.

Although several articulated “wristed” instruments have been proposed using rigid mechanical transmission (U.S. Pat. Nos. 5,330,502, 7,819,894, 7,674,255), flexible mechanical transmission is considered by many to exhibit better performance characteristics in terms of weight, friction and other attributes (WO9743942, U.S. Pat. Nos. 6,394,998, 6,554,844).

When metallic ropes are used with a suitable strand construction, flexible mechanical transmission can provide a fairly good axial stiffness with an acceptable radial (bending) flexibility. However, the life of the metallic ropes used in instruments employing flexible mechanical transmission is strongly affected by the value of the maximum tension to which they are exposed during their normal use. When metallic ropes are passed around pulleys, their constituent strands are forced to rub against each other, increasing the friction on the overall system, thus impacting mechanical transmission and causing the ropes to wear during several cycles of utilization. Therefore, the higher the tension on the ropes, the higher the friction on the system and the shorter the life of the instrument. Metallic ropes in pulley-driven systems can also be subject to stretching over time, thus resulting in a progressive reduction in actuation force at the end-effector over time. These considerations relating to friction, cable wear and cable stretching must be acknowledged in view of the mechanical constraints of cable-driven mechanical systems with pulleys, in which the force applied to system cables is not necessarily reflected at the end effector, typically being reduced as a function of the number of pulleys and links in the system. This phenomenon is described in greater detail in the following paragraphs with reference to a prior disclosure by the present applicants.

In the present applicants' previous disclosure, a cable-driven surgical instrument 120, has a main shaft 121 that allows the passage of flexible elements 124, 125, 126 that are able to transmit motion to three different end-effector links 127, 128, 129, from the proximal hub 123 at the articulated end-effector 122 of the instrument 120 (FIGS. 23 and 24 hereto).

As can be seen in FIGS. 25 and 26 hereto, the distal end-effector members 128, 129 are operatively connected to flexible members 125 and 126 so that they can be independently rotated in both directions along the distal axis 130. Contact between the flexible elements and the distal end-effector elements is made by way of the end effector pulleys 128a, 129a (FIG. 27 hereto), which are part of (or rigidly attached to) the end-effector links 128, 129. Then, by the combination of rotations of the two distal end-effector links 128, 129, it is possible to actuate the surgical instrument 120 in order to accomplish its function (FIG. 28 hereto).

An issue with the aforementioned system is related to the fact that the actuation forces applied at the tip of the instrument jaws are only a fraction of the forces to which the cables are exposed. This phenomenon is explained in FIG. 29 hereto, comprising a free body diagram of one of the distal end-effector members 129, applying a force F, measured at a point two thirds of the way to the distal end of its blade length, on a body 131. By considering the equilibrium of torques at the axis of rotation 130 and, for instance, a ratio of L/R=3 (wherein L is the distance from the axis of rotation 130 to the point of measurement of the applied force F and R is the radius of the effector pulley 129a), the tension T in the cable will be three times higher than the force F at the tip. This limitation can be problematic when high gripping forces are required at the distal end-effector tip of the instrument jaws 128, 129 (for instance, in needle holders). In cases such as these where high gripping forces are required, it is possible that enough force simply cannot be applied to the cables to achieve the necessary gripping force or, in the alternative, sufficient force can be applied but the resulting strain on the cables is too high, resulting in unacceptable wear or stretching as discussed above. Using the example of a needle holder, the forces applied at the proximal end of the instrument (provided by the hand of the user of by an actuator) have to be extremely high in order to avoid undesired movements of the needle (if sufficient force can be applied to avoid undesired movements), which can negatively impact the life of the instrument.

Accordingly, an aim of the present invention is to overcome the aforementioned drawbacks of known devices in certain articulated instrument applications by providing a new articulated end-effector mechanism, preferably to be used in a cable-driven surgical instrument. The new articulated end-effector mechanism should be capable of providing enough force to the instrument's distal jaws, especially when high actuation forces at the distal extremity of the instrument jaws are required and the usable life of the instrument has to be maximized. In addition, another aim of the present invention is to reduce the input forces required to actuate the instrument, resulting in more comfort to the user (if the instrument is fully mechanical) or less power required from the actuators (if the instrument if robotic).

### SUMMARY OF THE INVENTION

Theses aims and other advantages are achieved by a new articulated end-effector mechanism, designed to be used at the distal extremity of a surgical instrument shaft, in the form of, for example, a needle holder, scissor or grasper. The shaft defines the longitudinal axis of the instrument and is able to move according to the mobility constraints imposed by a body incision, which include a rotational movement about its own axis. This rotation also causes the rotation of the end-effector, mounted on the distal extremity of the shaft. Thus, the instrument shaft has the combined function of positioning the end-effector within the interior of the patient's body and allowing the passage of the different mechanical elements that are able to actuate the different distal end-effector articulations, by transmitting motion from the proximal extremity of the instrument shaft, to the distal end-effector articulations. These distal articulations of the end-effector are able to (1) actuate the surgical instrument in order to accomplish its function (for example, grasping or cutting) and (2) provide orientation motions between the end effector and the instrument shaft.

The actuation movement of each distal jaw of the end-effector is originated by an input movement on the proximal extremity of the instrument shaft, which is connected to a cam-and-follower mechanism, placed on the instrument's end-effector, by flexible transmission elements passing through the instrument shaft. This cam-and-follower mechanism is then able to transmit, and amplify, the force to a distal end-effector link (or jaw) by direct contact.

This mechanism is intended to be used primarily in surgical procedures, where the instruments with articulated end-effectors are passing through incisions into a patient's body. It is also adapted for any suitable remote actuated application requiring a dexterous manipulation with high stiffness and precision such as, but in no way limited to, assembly manipulation, manipulation in narrow places, manipulation in dangerous or difficult environments, and manipulation in contaminated or sterile environments.

### BRIEF DESCRIPTION OF FIGURES

The invention will be better understood according to the following detailed description of several embodiments with reference to the attached drawings, in which:

FIG. 1 shows a perspective view of a surgical instrument including an articulated end-effector according to an embodiment of the invention;

FIG. 2 shows a perspective view of an articulated end-effector of a surgical instrument according to an embodiment of the invention;

FIG. 3 shows the articulated end-effector of FIG. 2 in a first active position;

FIG. 4 shows the articulated end-effector of FIG. 2 in a second active position;

FIG. 5 shows the articulated end-effector of FIG. 2 in a third active position;

FIG. 6 shows the articulated end-effector of FIG. 2 in a fourth active position;

FIG. 7 shows the articulated end-effector of FIG. 2 in a sixth active position;

FIG. 8 shows the articulated end-effector of FIG. 2 in a seventh active position;

FIG. 9 shows a perspective view of the surgical instrument of FIG. 1 with a schematic cutout of an outer tube of the longitudinal shaft of the surgical instrument, through which is it possible to see the different flexible mechanical transmission elements;

FIG. 10 shows actuation topology for a distal end-effector link according to an embodiment of the invention;

FIG. 11 shows actuation topology for a second end-effector link according to an embodiment of the invention;

FIG. 12 shows actuation topology for a cam element of a cam-and-follower mechanism according to an embodiment of the invention;

FIG. 13 shows a side view of a cam-and-follower mechanism actuating a distal articulation of an instrument's end-effector according to an embodiment of the invention;

FIG. 14 shows the cam-and-follower mechanism of FIG. 13 in a first active position;

FIG. 15 shows the cam-and-follower mechanism of FIG. 13 in a second active position;

FIG. 16 illustrates the phenomenon of force amplification of a cam-and-follower mechanism with a single-pitch spiral-profile cam element according to an embodiment of the invention;

FIG. 17 illustrates the phenomenon of force amplification of a cam-and-follower mechanism with a dual-pitch spiral-profile cam element according to an embodiment of the invention;

FIG. 18 shows a perspective view of two cam elements (reverse and actuation) rigidly attached, according to an embodiment of the invention;

FIG. 19 shows a reverse cam-and-follower mechanism in a first active position according to the embodiment shown in FIG. 18;

FIG. 20 shows a reverse cam-and-follower mechanism in a second active position according to the embodiment shown in FIG. 19;

FIGS. 21 and 22 show an embodiment of the current invention with a spring element to reverse the actuation movement, in two different working positions;

FIG. 23 shows a perspective view of a surgical instrument previously disclosed by Applicants;

FIG. 24 shows a perspective view of an articulated end-effector of the surgical instrument shown in FIG. 23;

FIG. 25 shows the actuation topology for a first distal end-effector link of the surgical instrument shown in FIG. 23;

FIG. 26 shows the actuation topology for a second distal end-effector link of the surgical instrument shown in FIG. 23;

FIG. 27 shows a perspective views of the two distal end-effector links of the surgical instrument shown in FIG. 23;

FIG. 28 shows the articulated end-effector of the surgical instrument shown m FIG. 23 achieving an actuation by the movement of the distal end-effector links;

FIG. 29 shows a free body diagram of one of the distal end-effector members of the surgical instrument shown in FIG. 23.

### DETAILED DESCRIPTION OF THE INVENTION

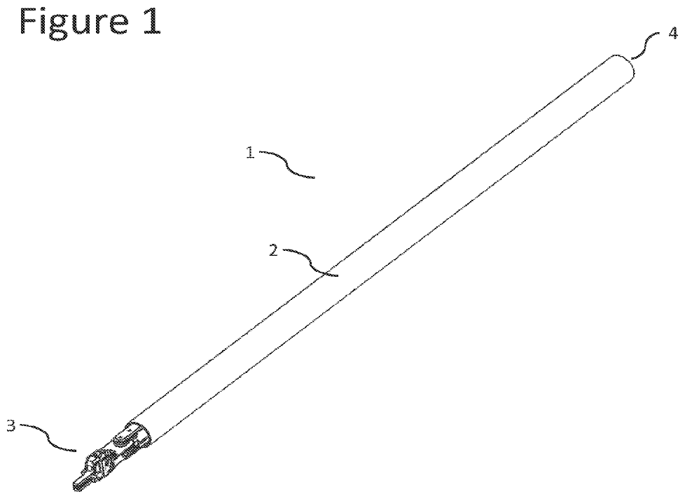

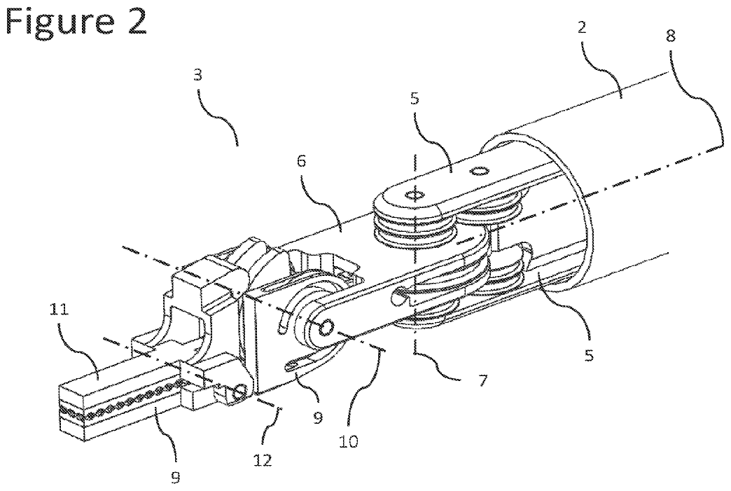

With general reference to FIG. 1, a surgical instrument 1 for minimally invasive surgical procedures, with an articulated end-effector constructed in accordance with an embodiment of the present invention, is described herein. This instrument 1 includes a main shaft 2 with a distal end-effector 3 and a proximal extremity 4 or head. Referring to FIG. 2, the end-effector 3 is connected to the distal extremity 20 of the main shaft 2 by a proximal joint, which allows the rotation of a proximal end-effector link 6 around a proximal axis 7 in such a manner that the orientation of the proximal end-effector link 6 with respect to the main shaft axis 8 can be changed.

Referring to FIG. 2, a second end-effector link 9 is rotatably connected to the proximal end-effector link 6 by a second end-effector joint, which is represented by the second end-effector axis 10. This second end-effector axis 10 is substantially perpendicular and non-intersecting with the proximal axis 7 and substantially intersects the main shaft axis 8.

Referring to FIG. 2, the distal end-effector link 11 is rotatably connected to the second end-effector link 9 by a distal end-effector joint, which is represented by the distal end-effector axis 12. This distal end-effector axis 12 is substantially parallel to the second end-effector axis 10 and perpendicular and non-intersecting with the proximal end-effector axis 7.

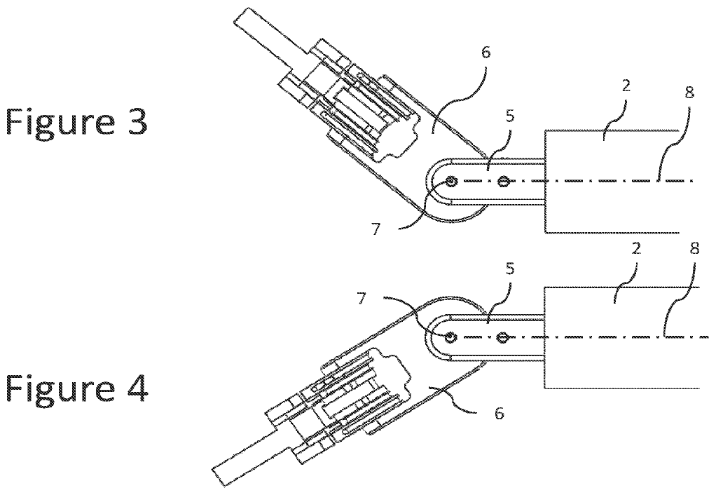

By actuating the proximal joint, the proximal end-effector link 6 can be angulated over the proximal axis 7, in the range of up to ±90°, with respect to the plane containing the main shaft axis 8 and the proximal axis 7, thus providing a first orientational degree of freedom for the end effector 3. FIGS. 3 and 4 show a surgical instrument 1 according to an embodiment of the present invention with different angular displacements at the proximal joint.

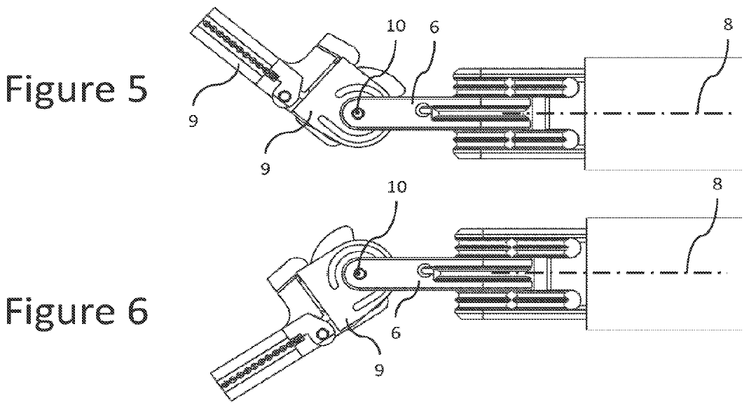

By actuating the second end-effector joint, the second end-effector link 9 can be angulated, substantially up to ±90°, over the second end-effector axis 10, with respect to the plane containing the main shaft axis 8 and the second end-effector axis 10, thus providing a second orientational degree of freedom for the end effector 3 that is perpendicular to the aforementioned first orientational degree of freedom. FIGS. 5 and 6 show a surgical instrument 1 according to an embodiment of the present invention with different angular displacements at the second end-effector joint.

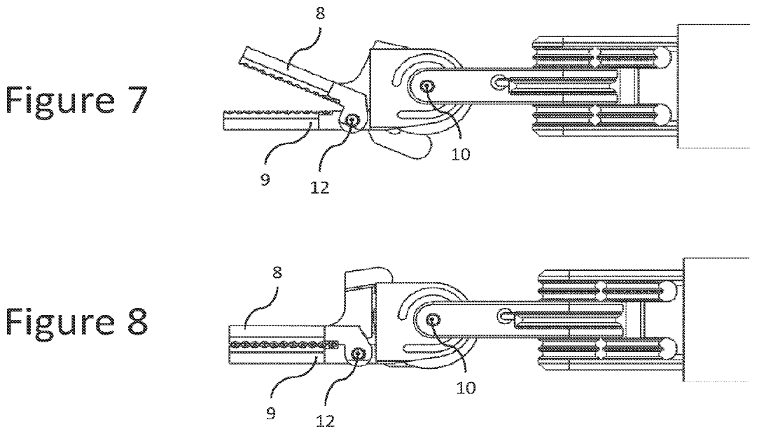

By actuating the distal end-effector joint, the distal end-effector link 11 can be angulated, over the distal end-effector axis 12, so that the surgical instrument is actuated in order to accomplish its function (for instance as a needle holder, scissors or forceps), thus providing an actuation degree of freedom at the end effector 3. FIGS. 7 and 8 show the surgical instrument 1 with different angular displacements at the distal end-effector joint.

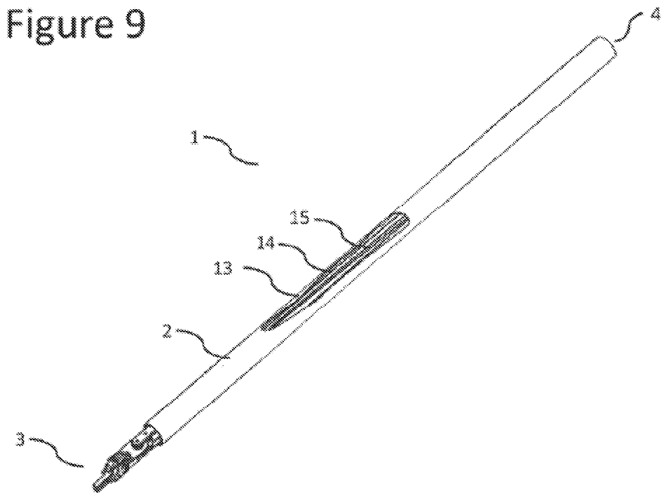

With reference to FIG. 9, the main shaft 2 allows the passage of flexible elements 13, 14, 15 that are able to deliver motion to the different end-effector links 6, 9, 11, from the proximal extremity 4 or head of the instrument shaft 2. The flexible elements 13, 14, 15, may optionally take the form of metal ropes or cables which may be constructed of tungsten, steel or any other metal suitable for surgical applications.

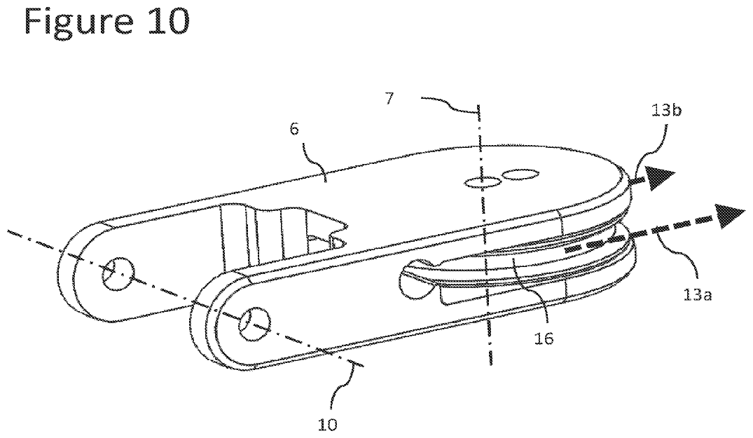

As can be seen in FIG. 10, the flexible element 13 comprises two different segments, 13a, 13b, which form a closed cable loop between the proximal end-effector link 6 and an input element at the proximal extremity 4 of the instrument shaft 2. The proximal end-effector link 6 is operatively connected to the flexible members 13a and 13b so that it can be independently rotated in both directions along the proximal axis 7. The contact between the flexible elements 13a, 13b and the proximal end-effector link 6 is made in a grooved pulley 16, which is rigidly attached or operably connected to the proximal end-effector link 6.

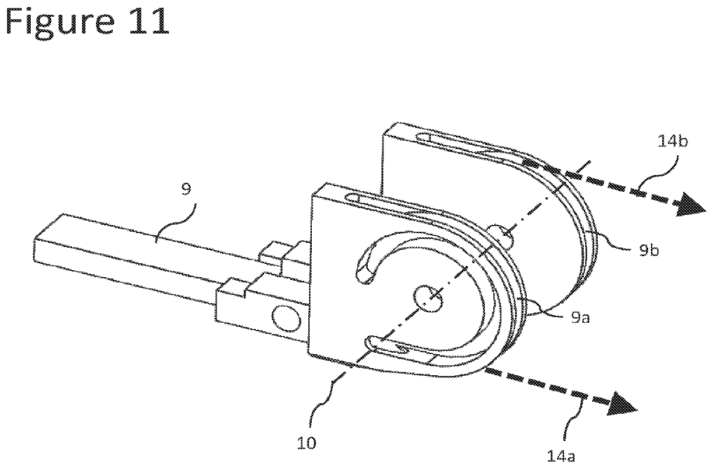

As can be seen in FIG. 11, the flexible element 14 comprises two different segments, 14a, 14b, which form a closed cable loop between the proximal end-effector link 6 and an input element at the proximal extremity 4 of the instrument shaft 2. The second end-effector link 9 is operatively connected to the flexible members 14a and 14b so that it can be independently rotated in both directions along the second end-effector axis 10. The contact between the flexible elements 14a, 14b and the second end-effector link 9 is made in the grooved surfaces 9a, 9b, which have a pulley-like geometry and are part of the second end-effector link 9.

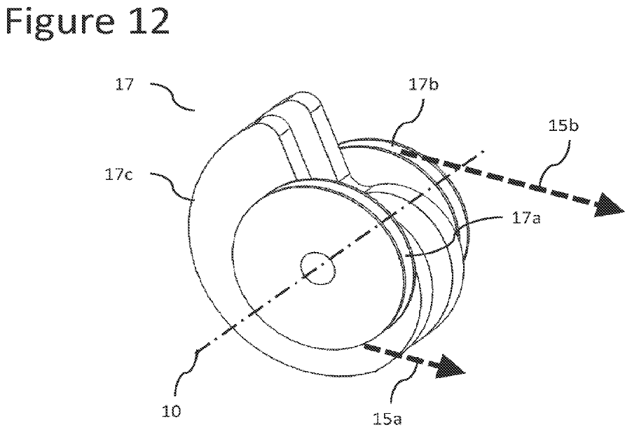

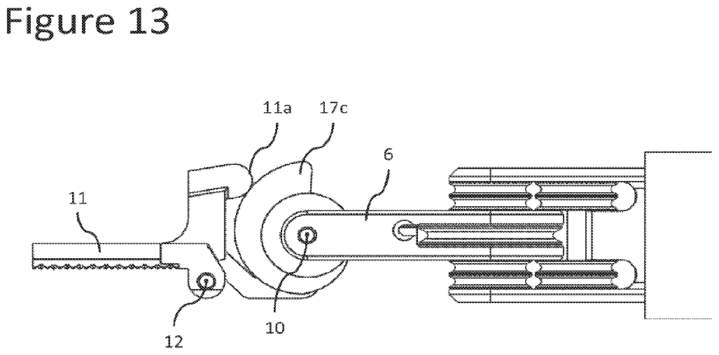

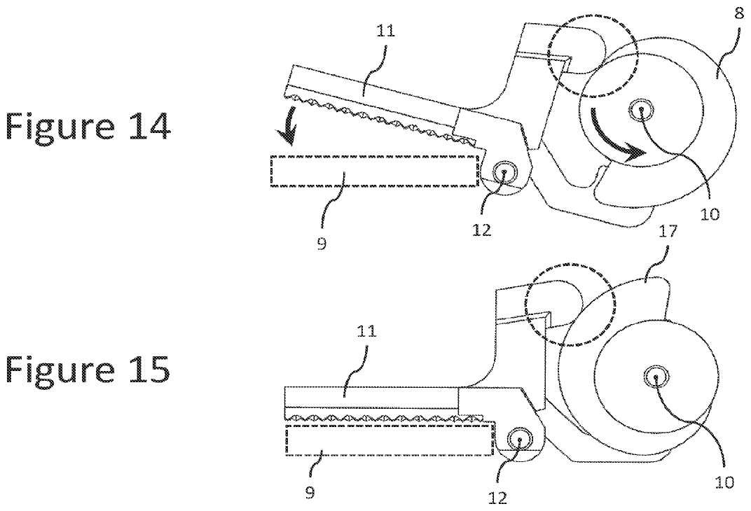

In order to increase the actuation (or gripping) force at the distal jaws 9, 11, while decreasing the tension in the flexible transmission elements, a cam-and-follower mechanism is used at the instrument's articulated end-effector 3. It comprises a cam element 17 (FIG. 12), having 2 grooved surfaces 17a, 17a, with pulley-like geometry, to which the flexible members 15a and 15b are attached, so that it can be independently rotated in both directions along the second end-effector axis 10. Rigidly attached or operably connected to these pulley-like geometrics 17a, 17b (or components), a cam-profile geometry 17c (or component) is also able to rotate in both directions along the second end-effector axis 10. Another element of the cam-and-follower mechanism is the follower geometry 11a (or component), which is part of (or rigidly attached to) the distal end-effector link 11 (FIG. 13). By being in contact with the cam-profile geometry 17c of the cam element 17, the follower geometry 11a (and therefore, necessarily, the distal end-effector link 11) is driven to rotate against the second end-effector element 9 when the cam element 17 is rotating (shown in counterclockwise rotation in FIGS. 14 and 15). This movement of the distal jaws 9, 11 moving against each other corresponds to the actuation of the surgical instrument 1, wherein the actuation force can be maximized by a careful selection of the profile of the cam element 17.

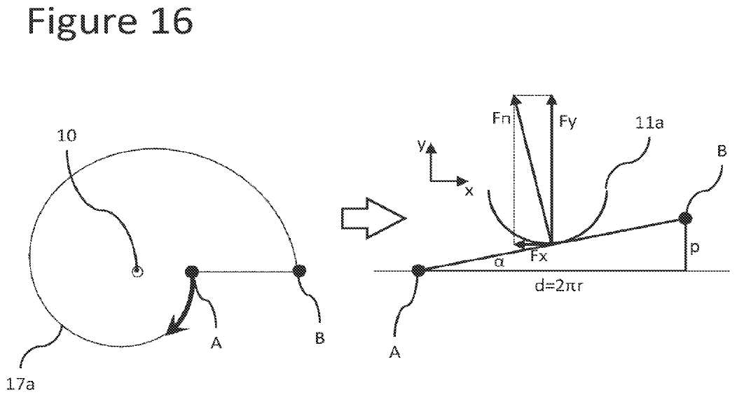

In some embodiments of the current invention, by way of example but not limitation, the cam element 17 may have a spiral profile (FIG. 16), whose rotation is able to drive the movement of the follower geometry 11a or component with a force that is much higher than the tension in the flexible element 15 that is driving the rotation. As a consequence, the instrument will be able to deliver high actuation forces at the jaws, while keeping the tension in the cables at more minimal values, which increases the fatigue performance and available usage cycles of the instrument and decreases the overall friction in the system.

This aforementioned force multiplication phenomenon can be better understood with the example of the wedge analogy of FIG. 16. With reference to the above embodiment, the rotation of the spiral cam element 17 so that the point of contact with the follower geometry 11a or component is traveling from point A to point B, is equivalent to driving along a γ vector a follower geometry 11a or component by moving a wedge along an x vector and having the point of contact travelling from point A to point B. The angle α of the wedge is optimally a function of the pitch of the spiral and its initial radius. The smaller the angle of the wedge, the higher the multiplication of forces, from cable tension to actuation force. Thus, variation of the wedge angle (by varying spiral pitch and initial spiral radius) can be used to ultimately control the degree of force multiplication and, consequently, the degree of reduction in cable tension.

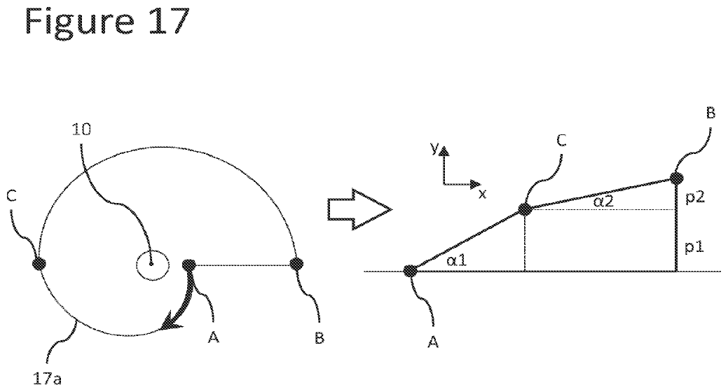

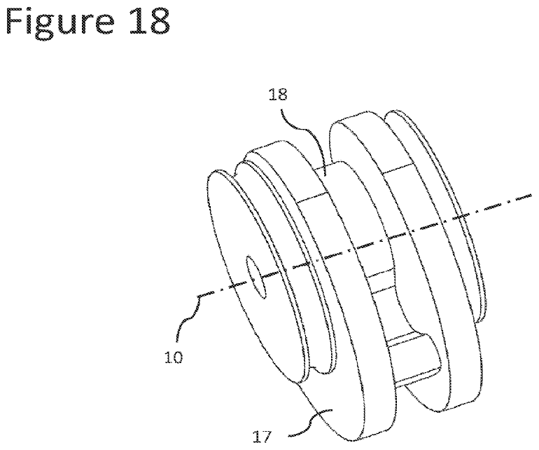

FIG. 17 shows an alternate embodiment of the current invention, where the cam profile 17a comprises different spiral profiles (from A to C and from C to B), with different pitches p1, p2. In the same way, in other embodiments of the current invention, a wide variety of shapes and profiles can be used in the cam element 17 to drive the follower geometry 11a to move according to different movement and force patterns.

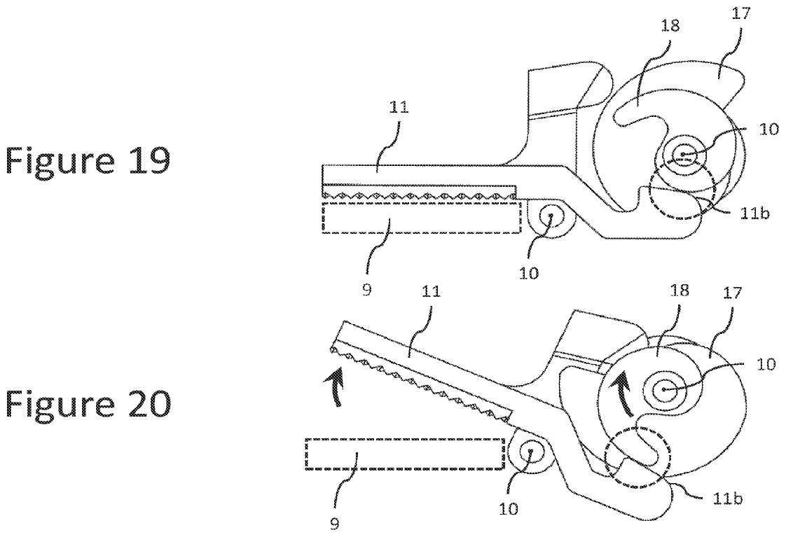

In a further alternate embodiment, and in order to reverse the movement of the jaws, a second cam-and-follower mechanism can be used. FIG. 18 shows how a reverse cam element 18 can be fixed to the actuation cam element 17 so that both cam profiles are able to rotate about the same axis 10. By being in contact with the cam element 18, the follower geometry 11b (and therefore the distal end-effector link 11) is driven to rotate away from the second end-effector element 9 when the cam element is rotating (shown rotating in a clockwise direction in FIGS. 19 and 20).

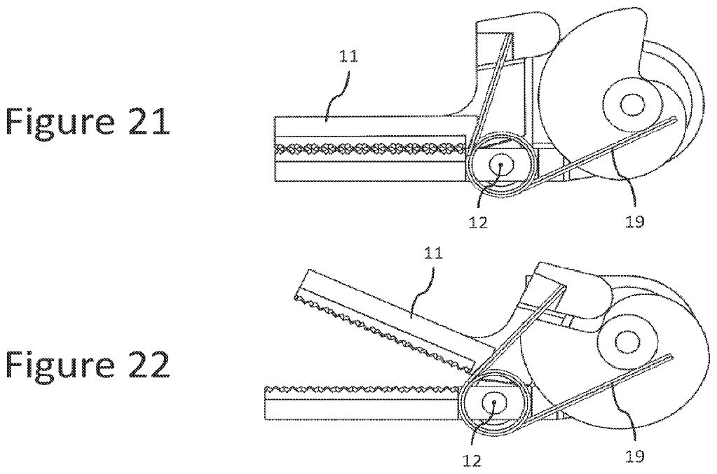

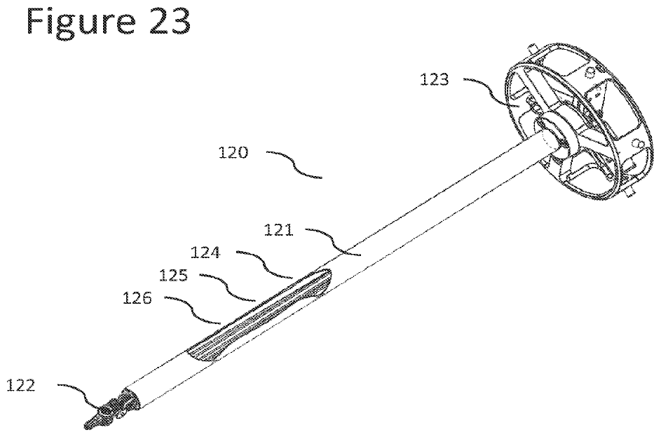

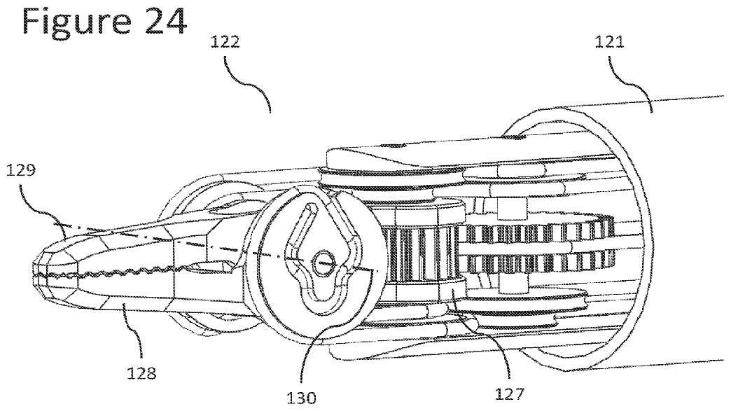

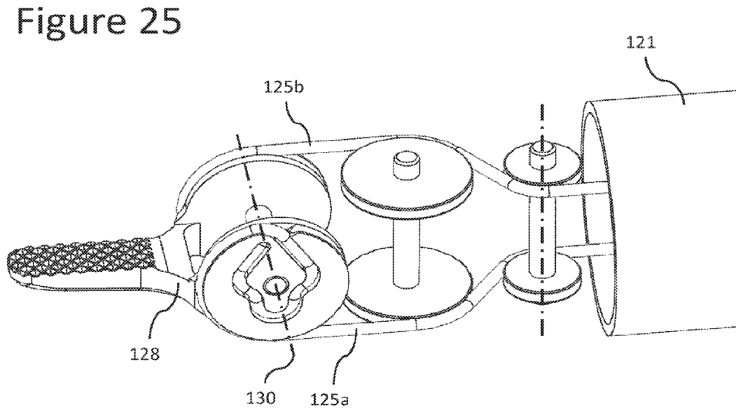

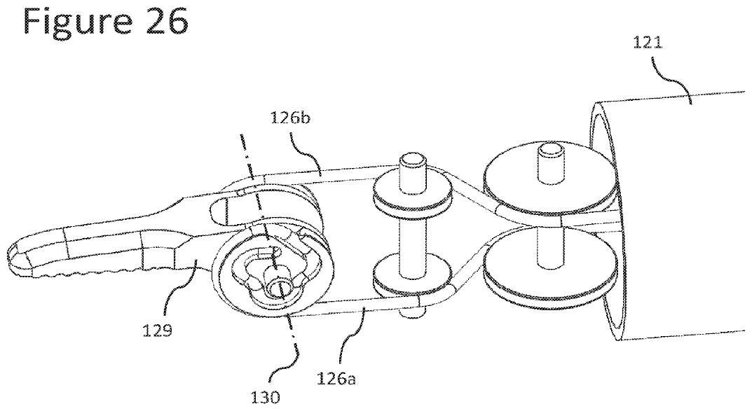

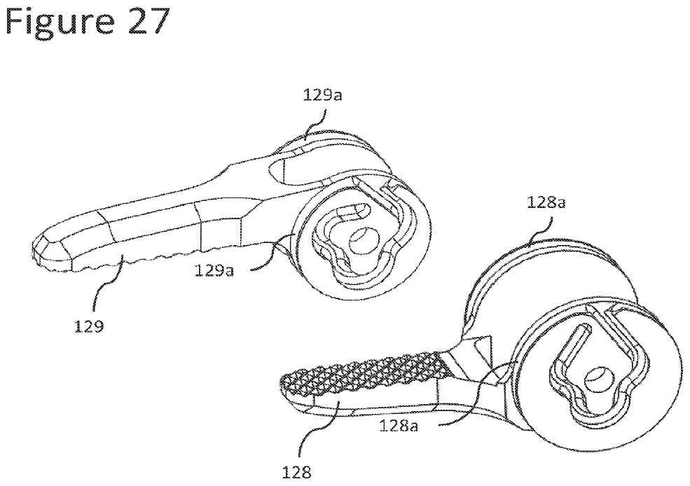

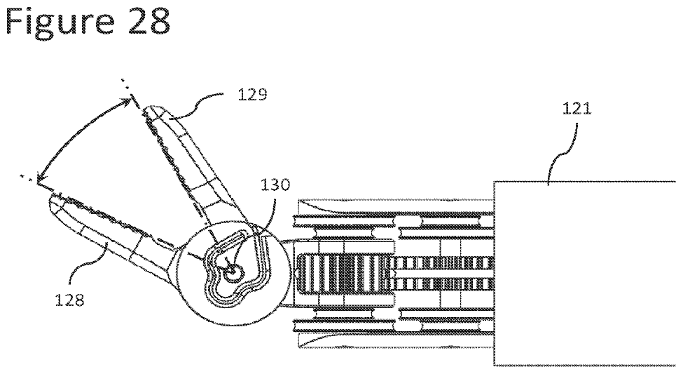

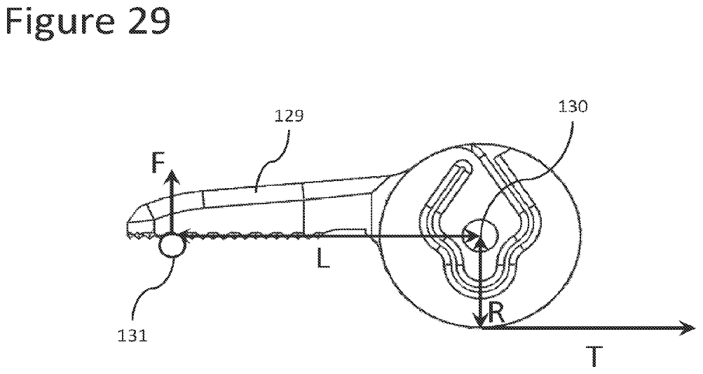

In yet another embodiment of the current invention, the reverse movement can be achieved not by a second cam-and-follower mechanism but by a spring element 19, which is able to rotate (about the axis 12) the distal end-effector link 11 back to its open position, when the cam element 17 rotates back (shown rotating clockwise in FIGS. 21 and 22) and the follower geometry 11a loses contact with the cam-profile geometry 17c of the cam element 17.

While this invention has been shown and described with reference to particular embodiments thereof, one of skill in the art will readily realise that various changes in form and details will be possible without departing from the spirit and scope of the invention as defined by the appended claims. Solely by way of example, one of skill in the art will understand that various geometries are possible for the cam-and-follower elements and that various angles are possible for the wedge element, thus impacting the force multiplication effect of the inventive system.

What is claimed is:

1. An articulated surgical instrument comprising:
an end-effector comprising one or more end-effector links, the one or more end-effector links comprising first and second follower geometries;
a first cam having a first spiral profile rotatably coupled to the first follower geometry;
a second cam having a second spiral profile rotatably coupled to the second follower geometry; and
flexible mechanical transmissions coupled to the first and second cams, the flexible mechanical transmissions configured to independently rotate the first and second cams in both directions about an axis of the first and second cams to thereby actuate the end-effector with an increased actuation force while reducing tension on the flexible mechanical transmissions via the one or more follower geometries.

2. The articulated surgical instrument of claim 1, further comprising two grooved surfaces coupled to the first and second cams, the two grooved surfaces configured to receive first and second flexible mechanical transmission to independently rotate the first and second cams in both directions about the axis of the first and second cams.

3. The articulated surgical instrument of claim 1, further comprising a longitudinal instrument shaft having a lumen configured to receive the flexible mechanical transmissions.

4. The articulated surgical instrument of claim 3, further comprising a proximal extremity, and wherein the flexible mechanical transmissions are configured to transmit motion from the proximal extremity to the end-effector through the lumen of the longitudinal instrument shaft.

5. The articulated surgical instrument of claim 1, wherein a first end-effector link of the one or more end-effector links is rotatably coupled to a second end-effector link of the one or more end-effector links via an end-effector joint.

6. The articulated surgical instrument of claim 5, wherein the end-effector joint is positioned distal to the first and second follower geometries and the first and second cams.

7. The articulated surgical instrument of claim 1, wherein the increased actuation force at the end-effector is higher than the reduced tension on the flexible mechanical transmissions.

8. The articulated surgical instrument of claim 1, wherein the reduced tension on the flexible mechanical transmissions increases fatigue performance of the articulated surgical instrument.

9. The articulated surgical instrument of claim 1, wherein the reduced tension on the flexible mechanical transmissions increases usage cycles of the articulated surgical instrument.

10. The articulated surgical instrument of claim 1, wherein the reduced tension on the flexible mechanical transmissions decreases overall friction of the articulated surgical instrument.

11. The articulated surgical instrument of claim 1, wherein rotation of the first and second cams by the flexible mechanical transmissions is configured to drive movement of the first and second follower geometries along the first and second spiral profiles of the first and second cams.

12. The articulated surgical instrument of claim 1, wherein the flexible mechanical transmissions comprise at least one of cables or metal ropes.

13. The articulated surgical instrument of claim 1, wherein variances in any of spiral pitch, initial spiral radius and spiral angle of the first and second spiral profiles influence a degree of the increased actuation force at the end-effector.

14. The articulated surgical instrument of claim 1, wherein the one or more end-effector links provide at least one orientational degrees of freedom and at least one actuation degree of freedom.

15. A method of actuating an end-effector of an articulated surgical instrument, the method comprising:
actuating flexible mechanical transmissions to independently rotate a first cam comprising a first spiral profile rotatably coupled to a first follower geometry of one or more end-effector links of the end-effector and a second cam comprising a second spiral profile rotatably coupled to a second follower geometry of one or more end-effector links of the end-effector to thereby drive movement of the first and second follower geometries along the first and second spiral profiles of the first and second cams and actuate the end-effector with an increased actuation force while reducing tension on the flexible mechanical transmissions via the first and second follower geometries.

16. The method of claim 15, wherein the increased actuation force is higher than the reduced tension on the flexible mechanical transmissions.

17. The method of claim 15, wherein actuating flexible mechanical transmissions to independently rotate the first and second cams comprises actuating first and second flexible mechanical transmissions received by two grooved surfaces coupled to the first and second cams.

18. The method of claim 15, wherein actuating the flexible mechanical transmissions to independently rotate the first and second cams increases fatigue performance of the articulated surgical instrument.

19. The method of claim 15, wherein actuating the flexible mechanical transmissions to independently rotate the first and second cams actuates the end-effector in one or more orientational degrees of freedom or one or more actuation degrees of freedom.

20. The method of claim 15, further comprising varying any of spiral pitch, initial spiral radius and spiral angle of the first and second spiral profiles to influence a degree of increased actuation force at the end-effector.
